(19) United States
(12) Patent Application Publication
(10) Pub. No.: US 2004/0064079 A1
(43) Pub. Date: Apr. 1, 2004

(54) Transcutaneous hemodialysis access system

(76) Inventors: Potito U. De Paolis, Los Angeles, CA (US); Saleh Salehmoghaddam, Los Angeles, CA (US)

Correspondence Address: Alan C. Rose, OPPENHEIMER WOLFF & DONNELLY LLP, Suite 3800, 2029 Century Park East, Los Angeles, CA, 90067, US

(21) Appl. No.: 10/261,555

(22) Filed: Sep. 30, 2002

(51) Int. Cl. A61M 37/00; A61M 31/00

(52) U.S. Cl. 604/5.01; 210/645

(57) ABSTRACT

The present invention discloses a vascular access device for hemodialysis, particularly, a transcutaneous access device. The transcutaneous access is optionally connected to one or more subcutaneous blood tubing and one or more sterile connectors which attach to the device and keeps the device sterile from external contamination when not in use. Advantages of the transcutaneous access include the elimination of the need to apply local anesthetic to the patient in order locate the access, the unnecessary bleeding or bruising of the patient and the reduction of total time of the dialysis procedure.

### BACKGROUND OF THE INVENTION

1. Field of Invention

This invention relates generally to a transcutaneous hemodialysis access system.

Specifically, the present invention identifies and describes a transcutaneous hemodialysis access system to eliminate the need for local anesthetic injections in order to locate the access.

2. Description of the Related Art

About 300,000 persons in the United States receive some sort of dialysis. A general description of dialysis is given in A. R., Nissenson & R. N. Fine, “Dialysis Therapy,” (3d ed., Hanley & Belfus, Inc., Philadelphia, Pa., 2002), incorporated herein by this reference.

A method to ensure blood access is important because blood is continually removed from and returned to the patient's body during hemodialysis. Typically, accesses are either vascular or peritoneal. For peritoneal dialysis, there is only one option, the placement of a catheter in the peritoneal cavity. There are several vascular access options available for hemodialysis, including arteriovenous (AV) fistula, synthetic graft or a silastic catheter.

Cuffed silastic catheters is one method of vascular access. Catheters are tunneled through the skin (subcutaneously) and inserted into the jugular or subclavian vein in the neck region. Unlike the longer-term AV fistulas and synthetic grafts, catheters are most often used on a short-term or temporary basis, or they may be used long-term in patients in whom an AV fistula or graft is not an option. A major disadvantage of silastic catheters is increased incidences of infection at the site of the catheter.

The most common vascular access method is AV fistulas. However, this approach is only good if patient's veins are large and there is time to create the fistula. Disadvantages of AV fistulas are that some patients with heart disease may have difficulties due to the increased blood flow and cardiac demands produced. Also, creating an AV fistula means choosing a type of anastomosis and site which will preserve the patient's native vessels. Thus, AV fistulas are not a long-term answer for blood access. Also, once the fistula needle is removed from the AV fistulas there is bleeding.

To date, the most long-term option is synthetic grafts. The subcutaneous graft is normally surgically placed in the forearm and provides the site to connect needles. Disadvantages of synthetic grafts include increased blood flow and cardiac demands on those patients with heart disease. Also, patients with synthetic grafts have increased incidences of thrombosis.

Another disadvantage of AV fistulas and synthetic grafts (the two most common methods) is that local anesthetics are required to create either the AV fistula or create a blood access in the graft. Typically, the technician/nurse first has to inject the patient with a local anesthetic to numb the skin. After several injections of a local anesthetic the technician/nurse then attempts to create the access by puncturing first the skin to locate the artery or vein in the case of creating an AV fistula, or in the case of a graft the needle has to be placed in the graft. The patient often endures multiple attempts or skin punctures before successful access is created. Multiple needle punctures into the skin not only causes pain but undue skin bruising to the patient. Then at the end of the dialysis treatment, removal of the needles from the fistulas also causes undue bleeding.

In short, the patient is subjected to unnecessary pain and bruising each time they have to have a dialysis treatment. Further, the typical patient receiving dialysis three times a week endures these procedures 12 times a month or 624 times per year. Therefore, improved methods to allow easy and efficient vascular access will improve the quality of life of the dialysis patient.

### INVENTION SUMMARY

A general object of the present invention is to provide a method of vascular access.

In accordance with one aspect of the present invention, these and other objectives are accomplished by providing a transcutaneous access.

In accordance with another aspect of the present invention, these objectives are accomplished by providing a method, which provides for a disposable connector attached to the access.

In accordance with another aspect of the present invention, these and other objectives are accomplished by providing a transcutaneous access and/or connector, which remains sterile during non-use.

In accordance with another aspect of the present invention, these objectives are accomplished by providing a transcutaneous access or connector to the access fabricated of flexible materials and does not create an immune response.

In accordance with another aspect of the present invention, these objectives are accomplished by providing a method of dialysis access, which does not require the use of local anesthetic.

In accordance with another aspect of the present invention, these objectives are accomplished by providing a method of dialysis access, which prevents bleeding after the needles are removed from the access.

In accordance with another aspect of the present invention, these objectives are accomplished by providing a method of long-term or short-term dialysis access.

In accordance with another aspect of the present invention, these objectives are accomplished by providing a method, which reduces the total amount of time of the dialysis treatment.

The above described and many other features and attendant advantages of the present invention will become apparent from a consideration of the following detailed description when considered in conjunction with the accompanying drawings.

### DETAILED DESCRIPTION OF THE PREFERRED EMBODIMENTS

This description is not to be taken in a limiting sense, but is made merely for the purpose of illustrating the general principles of the invention. The section titles and overall organization of the present detailed description are for the purpose of convenience only and are not intended to limit the present invention.

In hemodialysis it is very important to maintain a good vascular access so that blood can be removed and returned to a patient's body. Until recently, there have only been three vascular access options for patients: 1) arteriovenous (AV) fistulas; 2) synthetic grafts; and 3) catheters. Brief descriptions of these options and the disadvantages accompanying each are set forth above.

A new improved type of access is described in the present application. This improved type of access, called a transcutaneous hemodialysis access system, consists of a method providing for a device(s) that is transcutaneous and lies directly above the skin surface. The transcutaneous hemodialysis access device(s) can be directly connected to a native vessel or optionally the device(s) can be connected to at least one flexible subcutaneous tubing system.

A minor surgical procedure is performed to implant the access device(s). In a preferred embodiment of the present invention, the device(s) is incorporated with any existing blood tubing including synthetic grafts or any other subcutaneous blood tubing developed. For example, a graft may be surgically inserted with two branches or it may be inserted with two separate branches, and the access devices would be implanted in each branch of the graft. Alternatively, the access devices can be surgically implanted directly connected to an artery and/or a vein and thereby act as fistulas. If the access device(s) are connected directly to the native vessel then the device(s) can be anchored also by suturing them to nearby tissues. The device(s) can be placed anywhere subcutaneous blood tubing is inserted, for example, in the forearm, in the neck region or in the peritoneal cavity. Independent of where the blood tubing or native blood vessels are located, the access device(s) are typically directly above the skin surface and easy to locate.

Optionally, the needles or couplings from the dialysis apparatus, used for the transcutaneous hemodialysis access system will connect or couple to the access device(s) used. The needles or couplings are connected directly into the transcutaneous access lying above the surface of the skin. This method eliminates any need to use a local anesthetic to search for the subcutaneous access under the patient's skin. Elimination of such injections will cause virtually no pain or bruising to the patient.

Other advantages of the transcutaneous hemodialysis access system which are not provided for in a subcutaneous access system is that the total amount of time the patient now has to allocate for the treatment is substantially reduced since the technician/nurse does not spend time preparing the patient which involved searching for the subcutaneous access system in the first place.

Still another advantage of the transcutaneous hemodialysis access system is the elimination of bleeding when the needle is removed from the access because the needle does not have to be pulled out of the skin.

This improved transcutaneous hemodialysis access system also allows for increased movement during the dialysis depending on the location of the device(s) on the patient's body. For example, if it is placed near the neck region or the peritoneal cavity, the arms are free.

After dialysis treatment and the needle or couplings are removed from the access, the device(s) are cleaned, sterilized and a sterile bandage or armband is placed over the device(s) to maintain a sterile environment. There are various methods to keep the transcutaneous access device(s) sterile when not in use and any preferred method, which works, is sufficient. For example, in one preferred embodiment attaching a disposable connector to the transcutaneous hemodialysis access, prevents contamination because a new sterile connector replaces the used connector at the end of each dialysis session. Further, this sterile connector can be fabricated out of a flexible material so that it will bend when a bandage enclosure is wrapped around or on it. In another preferred embodiment, the disposable connector is closed and sealed and the seal is not broken until the next dialysis session when a needle or coupling is inserted or connected.

In another preferred embodiment, the device and the connector to said device is fabricated of materials which do not create immune responses in the patient's body, or on the surface of the skin.

In another preferred embodiment the device(s) can be used for long-term or short-term care of patients undergoing dialysis.

Accordingly, the invention is not limited to the precise embodiments described in detail hereinabove.

While the specification describes particular embodiments of the present invention, those of ordinary skill can devise variations of the present invention without departing from the inventive concept.

We claim:

1. A vascular access method for hemodialysis, comprising:
a) providing by a surgical procedure subcutaneous graft arrangements having a first branch connected to an artery and a second branch connected to a vein;
b) placing a first transcutaneous access device connected to the first branch of the graft which is further connected to an artery; and
c) placing a second transcutaneous access device connected to the second branch of the graft which is further connected to a vein; and
d) attaching connectors connected to the access devices thereby maintaining the device in a clean sterile environment; and
e) periodically coupling a hemodialysis apparatus to said first and second access devices.

2. A vascular access method as in claim 1 whereby the connectors are closed and sealed from the external environment until placement of a needle or coupling into the access devices.

3. A vascular access method as in claim 1 whereby the access devices and connectors are fabricated from flexible materials and the materials do not cause immune responses in the body or at the surface of the skin.

4. A vascular access method as in claim 1 whereby there is no local anesthetic used to insert the needle or coupling into the access devices or connectors.

5. A vascular access method as in claim 1 whereby there is no significant loss of blood after the needle or coupling is removed from the access devices or connectors.

6. A vascular access method as in claim 1 whereby the duration of the access devices provide for long-term or short-term blood access.

7. A vascular access method as in claim 1 whereby the total time from the start to the end of a dialysis session is significantly reduced.

8. A vascular access method for performance with a hemodialysis apparatus, comprising:
a) implanting by a surgical procedure a first transcutaneous access device connected to an artery; and
b) implanting by a surgical procedure a second transcutaneous access device connected to a vein.

9. A vascular access method as in claim 8 whereby any method which maintains the access devices in a sterile environment is used.

10. A vascular access method as in claim 8 whereby the access devices are fabricated from flexible materials and the materials do not cause immune response in the body or at the surface of the skin.

11. A vascular access method as in claim 8 whereby there is no local anesthetic used to insert the needle or coupling into the access devices.

12. A vascular access method as in claim 8 whereby there is no significant loss of blood after the needle or coupling is removed from the device.

13. A vascular access method as in claim 8 whereby the duration of the access devices provide for long-term or short-term blood access.

14. A vascular access method as in claim 8 whereby the total time from the start to the end of a dialysis session is significantly reduced.

15. A vascular access method for performance with a hemodialysis apparatus, comprising: providing a first and second transcutaneous access device connected to a first and second subcutaneous blood tubing and whereby the access devices are optionally connected to a first and second connector which maintains the devices in a sterile environment from external contamination until placement of the needle or coupling into the device or the device connector.

16. A vascular access method as in claim 15 whereby the connectors are closed and sealed and said connectors are fabricated of flexible materials.

17. A vascular access method as in claim 15 whereby there is no local anesthetic used to insert the needle or coupling into the device or connector.

18. A vascular access method as in claim 15 whereby removal of the needle or coupling from the device causes no significant bleeding.

19. A vascular access method as in claim 15 whereby the access devices provide for long-term or short-term blood access.

20. A vascular access method as in claim 15 whereby total time from the start to the end of a dialysis session is significantly reduced.
